(19) United States
(12) Patent Application Publication
(10) Pub. No.: US 2002/0086017 A1
(43) Pub. Date: Jul. 4, 2002

(54) GA binding protein and neurite derived growth factors for use in the treatment of muscular dystrophy

(76) Inventor: Tejvir S. Khurana, Pennsylvania, PA (US)

Correspondence Address: BIRCH STEWART KOLASCH & BIRCH, PO BOX 747, FALLS CHURCH, VA, 22040-0747, US

(21) Appl. No.: 09/852,068

(22) Filed: May 10, 2001

Related U.S. Application Data
(63) Continuation of application No. PCT/DK99/00694, filed on Dec. 9, 1999.

(30) Foreign Application Priority Data
Dec. 11, 1998 (DK) 1998 01639

(51) Int. Cl. A61K 39/395

(52) U.S. Cl. 424/146.1

(57) ABSTRACT

The present invention relates to methods and compositions for the treatment of muscular dystrophia, and in particular Duchenne's muscular dystrophy. More specifically the invention relates to GA Binding Protein, in particular GABPα and/or GABPβ, or neurite derived growth factors, in particular heregulin polypeptides, useful for the treatment of muscular dystrophy, in particular Duchenne's muscular dystrophy.

### CROSS REFERENCE

This application is a Continuation of PCT International Application No. PCT/DK99/00694 filed on Dec. 9, 1999, which was published in English and which designated the United States and on which priority is claimed under 35 U.S.C. §120, the entire contents of which are hereby incorporated by reference.

### TECHNICAL FIELD

The present invention relates to methods and compositions for the treatment of muscular dystrophia, and in particular Duchenne's muscular dystrophy. More specifically the Invention relates to GA Binding Protein, In particular GABPα and/or GABPβ, and neurite derived growth factors, in particular heregulin polypeptides, useful for the treatment of muscular dystrophy, in particular Duchenne's muscular dystrophy.

### BACKGROUND ART

Neurite derived growth factors are members of the neuregulin family of polypeptide growth factor homologues that includes heregulin, NDF, ARIA and GGF [Fischbach G D & Rosen K M: ARIA—A neuromuscular junction neuregulin; Annu. Rev. Neurosci. 1997 20 429-458]. These ligands and their receptors have wide ranging effects that are considered critical for nervous system development [Meyer D & Birchmeier C: Multiple essential functions of neuregulin in development; Nature 1995 378 386-3; Lemke G: Neuregulins in Development; Mol. Cell. Neurosci. 1996 7 247-262; Fischbach & Rosen, op cit.].

Heregulin designates a family of polypeptide activators of the HER/ErbB class of receptors and includes heregulin-α (HRG-(α), heregulin-β1 (HRG-β1), heregulin-β2 (HRG-β2) and heregulin β3 (HRG-β3), as described in WO 92/20798 and WO 98/35036. Also, heregulin variants have been described in WO 98/35036.

The neurite derived growth factors including heregulin have been suggested useful in the treatment of a variety of diseases including cancer. However, any effect on muscular dystrophia, and in particular Duchenne's muscular dystrophy, has never been reported.

Dystrophy is a term used to describe a variety of muscular diseases. A subgroup of muscular dystrophia happens to be caused by genetic defects. Among these, Duchenne's muscular dystrophy (DMD) is the most common neuromuscular disorder linked to the X-chromosome, and it is caused by genetic mutations that lead to quantitative and qualitative disturbances in the expression of dystrophin [Hoffman E P, Brown R H & Kunkel L M: Dystrophin: The protein product of the Duchenne muscular dystrophy locus; Cell 1987 51 919-928].

Utrophin shares extensive sequence homology and organisational motifs with dystrophin, and utrophin is considered to be the autosomal homologue of dystrophin [Love D R, et al.: An autosomal transcript in skeletal muscle with homology to dystrophin; Nature 1989 339 55-58; Tinsley J M, et al.: Primary structure of dystrophin-related protein; Nature 1992 360 591-593]. Moreover, utrophin is believed to share the functional properties with dystrophin [Khurana T S, Watkins S C, Chafey P, Chelly J, Tome F M, Fardeau M, Kaplan J C & Kunkel L M: Immunolocalization and developmental expression of dystrophin related protein in skeletal muscle; Neuromuscul. Disord. 1991 1 185-194]. The ability of high levels of utrophin to rescue dystrophin-deficient muscle was recently demonstrated by generating transgenic mice expressing high levels of utrophin and breeding them with dystrophin-deficient mdx mice [Tinsley J M, Potter A C, Phelps S R, Fisher R, Trickett J I & Davies K E: Amelioration of the dystrophic phenotype of mdx mice using a truncated utrophin transgene; Nature 1996 384 349-353].

There is currently no cure for DMD, and because of the many concerns associated with gene therapy, transgene mediated utrophin upregulation is not yet applicable to DMD-patients.

### SUMMARY OF THE INVENTION

According to the present invention it has now been found that neurite derived growth factors such as heregulin affect the regulation of utrophin gene expression with a subsequent enrichment of utrophin at the neuromuscular junction of skeletal muscle. Based on these findings, methods and compositions for the treatment of muscular dystrophia, and in particular DMD are provided.

The approach of utrophin upregulation is particularly attractive for the treatment of DMD, since utrophin (being autosomally encoded) is not affected by the chromosome Xp21 mutations that cause DMD. Because of the many concerns currently associated with gene therapy, transgene mediated utrophin upregulation is not directly applicable to DMD-patients, which makes the methods and compositions provided with this invention even more attractive.

Therefore, in its first aspect, the invention relates to the use of GA Binding Protein (GABP) for the manufacture of a medicament for the treatment of muscular dystrophia.

In a second aspect, the invention relates to the use of a neurite derived growth factor for the manufacture of a medicament for the treatment of muscular dystrophia.

In another aspect, the invention provides pharmaceutical compositions comprising a therapeutically effective amount of GABP or a neurite derived growth factor.

In another aspect, the invention relates to a method for treatment or alleviation of diseases, disorders or conditions relating to muscular dystrophia in a living body, said method comprising administering to said living body an effective amount of GABP or a neurite derived growth factor.

Other objects of the invention will be apparent to the person skilled in the art from the following detailed description and examples.

### DETAILED DISCLOSURE OF THE INVENTION

The present invention relates to methods and compositions for the treatment of muscular dystrophia, and in particular Duchenne's muscular dystrophy (DMD).

GA Binding Protein

GA Binding Protein (GABP) includes any GABP including, but not limited to, GABPα, GABPβ, a heterodimer complex of GABPα and GABPβ (GABPα/β), and GABP fusion proteins.

Neurite Derived Growth Factors

In its second aspect, the invention relates to the use of a neurite derived growth factor for the treatment of muscular dystrophia. The neurite derived growth factor for use according to the invention may in particular be heregulin, NDF, ARIA, or GGF.

In a preferred embodiment, the neurite derived growth factor for use according to the invention is an activator of the HER/ErbB class of receptors, preferably ErbB-2, ErbB-3, and/or ErbB-4.

In a more preferred embodiment, the neurite derived growth factor for use according to the invention is heregulin, a heregulin-like polypeptide, an isoform of heregulin, or a variant of heregulin.

In a more preferred embodiment, the neurite derived growth factor for use according to the invention is heregulin-α (HRG-α), heregulin-β1 (HRG-β1), heregulin-β2 (HRG-β2), heregulin β3 (HRG-β133), or γ-heregulin.

The neurite derived growth factor for use according to the invention may be obtained by any available route. In a preferred embodiment the neurite derived growth factor is a heregulin polypeptide obtained as described in International Patent Application WO 92/20798, a heregulin protein obtained as described in WO 96/16176, a heregulin protein obtained as described in WO 96/31599, a γ-heregulin obtained as described in WO 98/02541, a heregulin derivative obtained as described in WO 98/21956, or a variant of heregulin obtained as described in WO 98/35036.

Muscular Dystrophia

The present invention provides methods and compositions for the treatment of muscular dystrophia.

Dystrophy is a term used to describe a variety of muscular diseases. A subgroup of muscular dystrophia happens to be caused by genetic defects. Among these, Duchenne's muscular dystrophy (DMD) is the most common neuromuscular disorder linked to the X-chromosome, and it is caused by genetic mutations that lead to quantitative and qualitative disturbances in the expression of dystrophin [Hoffman E P, Brown R H & Kunkel L M: Dystrophin: The protein product of the Duchenne muscular dystrophy locus; Cell 1987 51 919-928].

Dystrophin, belongs to the spectrin superfamily of proteins, which includes the spectrins, the actinins and three close relatives of dystrophin, the chromosome 6-encoded dystrophin related protein (DRP) or utrophin [Love D R, et al.: An autosomal transcript in skeletal muscle with homology to dystrophin; Nature 1989 339 55-58; Khurana T S, Hoffman E P & Kunkel L M: Identification of a chromosome 6-encoded dystrophin-related protein; J. Biol. Chem. 1990 265 16717-16720; Tinsley et al., 1992, op cit.], the chromosome-18 encoded dystrobrevin [Khurana T S, Engle E C, Bennett R R, Silverman G A, Selig S, Bruns G A & Kunkel L M: (CA) repeat polymorphism in the chromosome 18 encoded dystrophin-like protein; Hum. Mol. Gen. 1994 3 841-841] and the chromosome-X encoded DRP-2 [Roberts R G. Freeman T C, Kendall E, Vetrie D L, Dixon A K, Shaw S C, Bone Q & Bobrow M: Characterization of DRP2, a novel human dystrophin homologue; Nature Gen. 1996 13 223-226]. In muscle, dystrophin is complexed to the membrane bound dystroglycan/sarcoglycan complex which forms a link with the extracellular matrix via laminin. Mutations in the genes encoding various members of the complex disrupt sarcolemmal integrity and result in a variety of X-linked and limb girdle muscular dystrophies [Brown R H: Dystrophin-associated proteins and muscular dystrophies; Annu. Rev. Med. 1997 48, 457-466; Campbell K P: Three muscular dystrophies: loss of cytoskeletal extracellular matrix linkage; Cell 1996 80 675-679].

These findings have led to methods and compositions for upregulation of utrophin in muscular diseases such as DMD, as well as methods and compositions by which neurite derived growth factors such as heregulin affect the regulation of utrophin gene expression and subsequent enrichment at the neuromuscular junction of skeletal muscle.

Based on these findings we provide methods and compositions for the treatment of muscular dystrophia, and in particular we provide pharmacological means to achieve utrophin upregulation in skeletal muscle of DMD-patients.

Biological Activity

Using a variety of molecular and cell biological techniques we have demonstrated that the growth factor heregulin increases de novo utrophin transcription in skeletal muscle cultures. Using DNA-affinity columns, immunoblots and EMSA we have identified the ets-related GABP-α/β complex as transcriptional mediators that bind and activate the utrophin promoter.

The utrophin promoter is a CpG rich promoter devoid of TATA or CAAT boxes [Dennis C L, Tinsley J M, Deconinck A E & Davies K E: Molecular and functional analysis of the utrophin promoter; Nucl. Acids Res. 1996 24 1646-1652]. This organisation is typically associated with housekeeping genes. However, while ubiquitously expressed, the utrophin gene is highly regulated at the level of developmental expression as well as sub-cellular distribution in both brain and muscle. In the brain, utrophin is highly enriched at the astrocytes forming the abluminal aspect of the blood-brain barrier, in close apposition to the extracellular matrix [Khurana T S, Watkins S C & Kunkel L M: The subcellular distribution of chromosome 6-encoded dystrophin-related protein in the brain; J. Cell. Biol. 1992 119 357-366; Khurana T S, Kunkel L M, Frederickson A D, Carbonetto S & Watkins S C: Interaction of chromosome-6-encoded dystrophin related protein with the extracellular matrix; J. Cell. Sci. 1995 108 173-185], while in skeletal muscle (an elongated multi-nucleated cell), utrophin protein is enriched at the NMJ/synapse [Khurana, T. S., Watkins, S. C., Chafey, P., Chelly, J., Tome, F. M., Fardeau, M., Kaplan, J. C. & Kunkel, L. M: Immunolocalization and developmental expression of dystrophin related protein in skeletal muscle; Neuromuscul. Disord. 1991 1 185-194; Nguyen T M, Ellis J M, Love D R, Davies K E, Gatter K C, Dickson G & Morris G E: Localization of the DMDL gene-encoded dystrophin-related protein using a panel of nineteen monoclonal antibodies: presence at neuromuscular junctions, in the sarcolemma of dystrophic skeletal muscle, in vascular and other smooth muscles, and in proliferating brain cell lines; J. Cell. Biol. 1991 115 1695-700; Ohlendieck K, Ervasti J M, Matsumura K, Kahl S D, Leveille C J & Campbell K P: Dystrophin-related protein is localized to neuromuscular junctions of adult skeletal muscle; Neuron. 1991 7, 499-508] and utrophin transcripts selectively accumulate in the post-synaptic sarcoplasmic compartment, in part, due to being preferentially expressed at the sub-synaptic nuclei rather than nuclei scattered along the length of the myofibre [Gramolini A O, Dennis C L, Tinsley J M, Robertson G S, Cadtaud J, Davies K E & Jasmin B J: Local transcriptional control of utrophin at the neuromuscular synapse; J. Biol. Chem. 1997 272, 8117-8120].

These local control mechanisms are reminiscent of those used by some of the nicotinic acetylcholine receptor (nACHR) subunit genes to help control the spatial distribution of ACHR in specific regions of the myofibre. nACHR genes are subject to regulation by contact (at the NMJ) with nerves and nerve-derived growth factors, e.g. ARIA/heregulin, Agrin, CGRP [Duclert A & Changeux J P: Acetylcholine receptor gene expression at the developing neuromuscular junction; Physiol. Rev. 1995 75 339-368; Fischbach & Rosen, op cit.]. The treatment of cultured muscle cells with heregulin results in dramatic increases in the rate of synthesis and accumulation of nACHR and sodium channels in the sarcolemma. The upregulation occurs via heregulin binding and activating the HER/ErbB class of receptor tyrosine kinases followed by activation of the P13 and MAP-Kinase pathways leading to an increase of nACHR subunit gene transcription. The activation of nACHR subunit gene transcription by neurite associated heregulin, seems to occur preferentially at the nuclei that lie immediately adjacent (and under) the synapse, rather than intramuscular nuclei that lie scattered along the length of the myofibre [Carraway K & Burden S J: Neuregulins and their receptors; Curr. Op. Neurobiol. 1995 5, 606-612; Tansey M G, Chu G C & Merlie J P: ARIA/HRG regulates the AChR e subunit gene expression at the neuromuscular synapse via activation of the phosphatidylinositol 3-kinase and RAS/MAPK pathway; J. Cell. Biol. 1996 134 465-476; Fischbach & Rosen, op cit.]. Additionally, the nACHR molecules are subject to post-translational modifications such as increased clustering by activation of the receptor tyrosine kinase MuSK by agrin [Glass D J et al.: Agrin acts via a MuSK receptor complex; Cell 1996 85 513-523]. A recent report has suggested the role of agrin in utrophin expression, via a yet to be identified pathway [Gramolini A O, et al.: Muscle and Neural Isoforms of Agrin increase utrophin expression in cultured myotubes via a transcriptional regulatory mechanism; J. Biol. Chem. 1998 273 736-743]. However, it is unclear if the effects noted in the study were indeed primarily due to agrin, or due to activation of HER receptors secondary to agrin treatment. Since the authors reported that both the neural and muscle isoforms of agrin increased transcription it is unlikely that agrin's effects on utrophin transcription occurred via the physiological (MuSK receptor) pathway for agrin, which is known to have discern between these isoforms [Glass et al., op cit.]. Experiments monitoring the activation of HER and MuSK receptors upon treatment with agrin need to be performed to resolve this issue in a satisfactory manner.

The presence of E-box, N-box and SP1 motifs in the utrophin promoter suggest that transcriptional factors may contribute to regulation of utrophin transcription [Dennis C L, Tinsley J M, Deconinck A E & Davies K E: Molecular and functional analysis of the utrophin promoter; Nuc. Acid Res. 1996 24 1646-1652]. The E-box is a binding site for helix-loop-helix proteins of the MyoD family and found in all nACHR subunit gene promoters, however, does not contribute to synapse-specific expression of the nACHRe-subunit gene [Duclert & Changeux, op cit.]. The N-box region has previously been shown to be critical for in vivo, synapse-specific expression of the nACHR d & e subunits [Duclert & Changeux, op cit.]. Recently, the heregulin response element of the ACHRe gene was mapped to a region that overlaps the N-box region [Sapru M K, Florance S K, Kirk C & Goldman D: Identification of a neuregulin and protein-tyrosine phosphatase response element in the nicotinic acetylcholine receptor e subunit gene: Regulatory role of an ets transcription factor; Proc. Natl. Acad. Sci. U.S.A. 1998 95 1289-1294]. Additionally, the N-box motif has been shown to be critical for mediating the effect of heregulin on ACHRe transcription, via ets-transcription factor GABP-α/β [Schaeffer L, Duclert N, Dymanus M H & Changeux J P: Implication of a multisubunit Ets-related transcription factor in synaptic expression of the nicotinic acetylcholine receptor; EMBO J. 1998 17 3078-3090]. Taken together it is evident that the 6 bp N-box (position −60 to −55 in the murine nACHRe gene) is contained within a larger 15 bp heregulin response element (position −69 to −55 in the murine nACHRe gene), and mutations removing the N-box in these experiments also abolished the GABP-α/β binding site in the nACHRe gene [Sapru et al., op cit.; Schaeffer et al., op cit.].

Our results show that a heregulin response element in the utrophin gene maps to the N-box region (position −206 to −201 of the utrophin gene, relative to transcription start site). Mutagenesis of the utrophin N-box in our constructs, also destroyed the GGA core of the ets-binding site (C/A GGA (A/T)) in this region of the utrophin promoter (5′ATCTTCcggaa C 3′ (SEQ ID NO: 7): N-box underlined, ets-domain italicised) due to the overlap of these motifs. This mutagenesis was accompanied with a loss of heregulin responsiveness, confirming the suggestion that this region of the utrophin promoter is critical for heregulin responsiveness. The ets-transcription factors often cooperate with other factors that bind and activate DNA elements in the vicinity leading to a myriad of mechanisms capable of achieving tight spatial and developmental control over subcellular expression. In the myeloid lineage SP1 has been shown to cooperate with GABP-αβ to activate the CD18 (2 leukocyte integrin) promoter [Rosmarin A G, Luo M, Caprio D G, Shang J & Simkevich C P: Sp1 cooperates with the ets transcription factor, GABP, to activate CD18(2 Integrin) promoter; J. Biol. Chem. 1998 273 13097-13103]. Similar cooperation may be operative for utrophin regulation in muscle as well, due to the coexistence of SP1 and ets-binding sites in the utrophin promoter. Additionally, the utrophin promoter may be subject to transcriptional downregulation by repressors recognising the ets site such as ERF or ERF-like molecules [Sgouras D N, Athanasiou M A, Beal G J. Fisher R J, Blair D G & Mavrothalassitis G J: ERF: an ets domain protein with strong transcriptional repressor activity, can suppress ets-associated tumorigenesis and is regulated by phosphorylation during cell cycle and mitogenic stimulation; EMBO J. 1995 14 4781-4793]. Thus active repression may be a mechanism involved in the sharp reduction of utrophin that occurs during the perinatal period and leads to the relatively low levels (typically 0.01% of message) of utrophin encountered in adult skeletal muscle [Khurana T S, Hoffman E P & Kunkel L M: Identification of a chromosome 6-encoded dystrophin-related protein; J. Biol. Chem. 1990 265 16717-16720]. Thus, heregulin may influence utrophin expression, by changing the relative levels/activity of transcriptional repressors as well as activators such as GABP-α/β complex.

In conclusion, we found that heregulin released from nerve terminals of motor neurons plays a role in controlling the enrichment of utrophin at the NMJ of skeletal muscle during development by transcriptional activation of the utrophin promoter via GABP-α/β. Based on these findings we provide methods and compositions for the treatment of muscular dystrophia, and in particular we provide pharmacological means to achieve utrophin upregulation in skeletal muscle of DMD-patients.

Pharmaceutical Compositions

In another aspect the invention provides novel pharmaceutical compositions comprising a therapeutically effective amount of GABP or the neurite derived growth factor of the invention.

While the GABP or neurite derived growth factor of the invention for use in therapy may be administered in the form of the raw chemical compound, it is preferred to introduce the active ingredient in a pharmaceutical composition together with one or more adjuvants, excipients, carriers, buffers, diluents, and/or other customary pharmaceutical auxiliaries.

In a preferred embodiment, the invention provides pharmaceutical compositions comprising the GABP or neurite derived growth factor of the invention together with one or more pharmaceutically acceptable carriers therefor, and, optionally, other therapeutic and/or prophylactic ingredients. The carrier(s) must be “acceptable” in the sense of being compatible with the other ingredients of the formulation and not harmful to the recipient thereof.

Pharmaceutical compositions of the invention may be those suitable for oral, rectal, bronchial, nasal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The GABP or neurite derived growth factor of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

Further details on techniques for formulation and administration may be found in the latest edition of Remington's Pharmaceutical Sciences (Maack Publishing Co., Easton, Pa.).

A therapeutically effective dose refers to that amount of active ingredient which ameliorates the symptoms or condition. Therapeutic efficacy and toxicity, e.g. ED₅₀ and LD₅₀, may be determined by standard pharmacological procedures in cell cultures or experimental animals. The dose ratio between therapeutic and toxic effects is the therapeutic index and may be expressed by the ratio LD₅₀/ED₅₀. Pharmaceutical compositions which exhibit large therapeutic indexes are preferred.

The dose administered must of course be carefully adjusted to the age, weight and condition of the individual being treated, as well as the route of administration, dosage form and regimen, and the result desired, and the exact dosage should of course be determined by the practitioner.

The actual dosage depend on the nature and severity of the disease being treated and the route of administration, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

Methods of Therapy

In another aspect the invention provides a method for the treatment or alleviation of muscular dystrophy, and which method comprises administering to such a living animal body, including a human, in need thereof an effective amount of GABP or a neurite derived growth factor. In a more preferred embodiment the invention provides a method a method for the treatment or alleviation of Duchenne's muscular dystrophy (DMD).

It is at present contemplated that suitable dosage ranges are 0.1 to 1000 milligrams daily, 10-500 milligrams daily, and especially 30-100 milligrams daily, dependent as usual upon the exact mode of administration, form in which administered, the indication toward which the administration is directed, the subject involved and the body weight of the subject involved, and further the preference and experience of the physician in charge.

### BRIEF DESCRIPTION OF THE DRAWINGS

The present invention is further illustrated by reference to the accompanying drawing, in which:

FIG. 1 shows the increase of utrophin mRNA in skeletal muscle cultures by heregulin. Differentiated L6 rat myotubes were incubated with 1 nM Heregulin in PBS for 30 minutes along with controls. RNA was extracted and quantitative RT-PCR performed. FIG. 1A shows the 322 bp Utrophin fragment and the 194 bp GAPDH control fragment obtained by RT-PCR (Untreated and Heregulin treated, respectively). FIG. 1B shows the results of radioactive quantification of four individual experiments taken together (Incorporated Radioactivuity (Normalized) vs. Untreated and HRG treated, respectively). The stippled bars represent Utrophin mRNA levels in untreated cells and cross hatched bars the levels in Heregulin (HRG) treated cultures. Heregulin treatment increases the endogenous utrophin message in muscle cell cultures to 195% of control levels (Error bars=s.e.m., n=4). Asterisks denote the results were statistically highly significant at p<0.001.

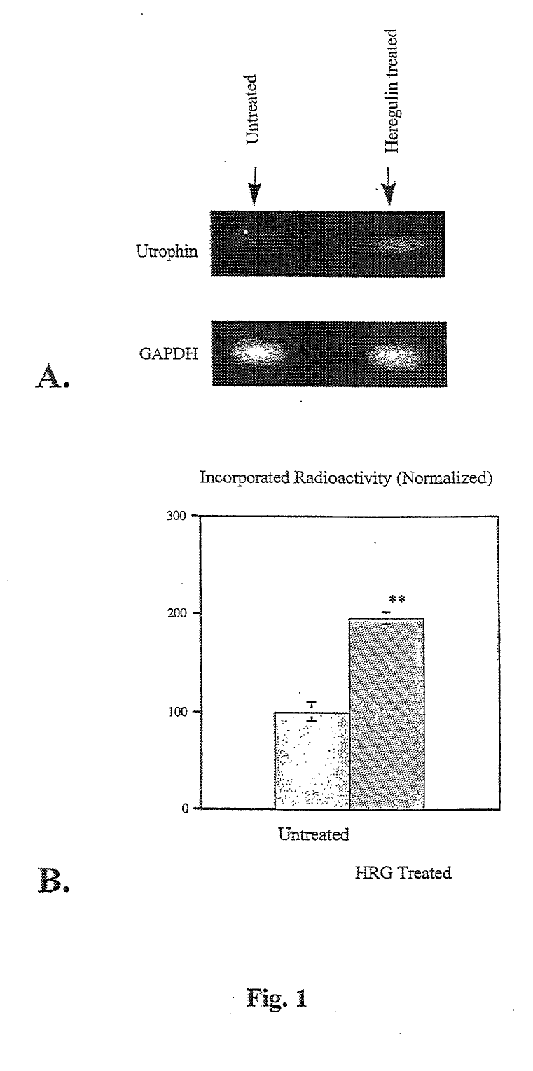

FIG. 2 shows that heregulin activates the Utrophin promoter in muscle cell cultures. The utrophin promoter luciferase-reporter construct pPUBF was co-transfected into L6 muscle cell lines or mouse primary muscle cultures along with transfection control plasmid pRL and assayed after 24-48 hrs of incubation with heregulin containing medium or control containing medium. FIG. 2A shows that pPUBF derived firefly luciferase activity is normalized to pRL derived renilla luciferase activity (internal control) as 100% in the untreated group, and expressed as luciferase activity (normalized) (Luciferase Activity (Normalized) vs. Untreated and HRG treated, respectively). Graph represents the summary of 10 individual experiments, 5 sets of experiments in primary mouse cultures and 5 sets of experiments in L6 rat muscle cell cultures. The stippled bars represent utrophin promoter activity in untreated cells and cross hatched bars the levels in heregulin (HRG) treated cultures. FIG. 2B shows a schematic of the pPUBF utrophin/luciferase construct [Dennis C L, Tinsley J M, Deconinck A E, & Davies K E: Molecular and functional analysis of the utrophin promoter; Nucl. Acids Res. 1996 24 1646-1652]. Heregulin increases de novo utrophin transcription in muscle cell cultures to 138% of control levels (Error bars=s.e.m., n=60). Asterisks denote the results were statistically highly significant at p<0.001.

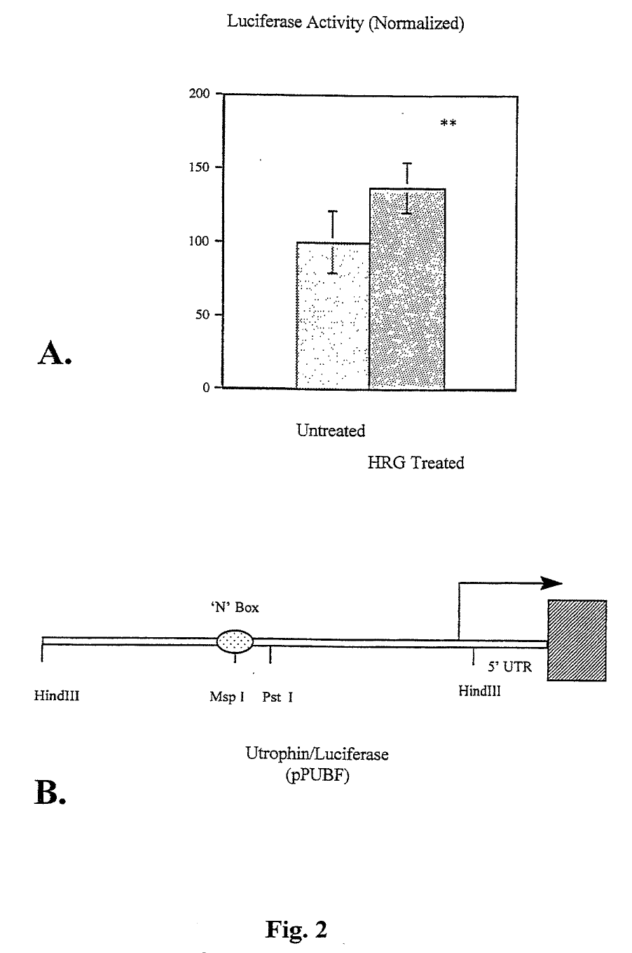

FIG. 3 shows that the Utrophin N-Box binds proteins in nuclear extracts of cultured L6 myotubes. Electrophoretic mobility shift assay was performed with the radiolabelled UtroNBox probe using 10 μg nuclear extract made from cultured L6 myotubes. Lane 1 shows the migration of free, unretarded probe control (Probe). Lane 2 demonstrates a mobility shift of the probe in the presence of a N-box binding factor present in L6 nuclear extracts (+Nuc Ext). Lane 3 is a control for specificity showing that the mobility shift was competed with a 1000× excess of unlabelled probe (+Nuc Ext; +1000×; Cold probe).

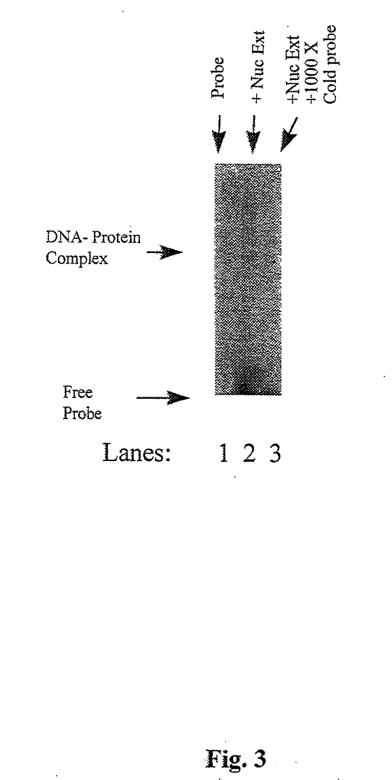

FIG. 4 shows that the N-Box motif in the utrophin promoter mediates transcriptional activation by heregulin in cultured muscle cells. The mutant utrophin promoter luciferase-reporter construct DNBox, (deleted in the N-box), was co-transfected into L6 muscle cell lines along with transfection control plasmid pRL and assayed after 24 hrs of incubation with either heregulin containing medium or controls. FIG. 4A shows that the DNBox derived firefly luciferase activity is normalized to pRL derived renilla luciferase activity (internal control) as 100% in the untreated state, and expressed as luciferase activity (normalized). The stippled bars represent mutant utrophin promoter activity in untreated cells and cross hatched bars the levels in heregulin (HRG) treated cultures (Luciferase Activity (Normalized vs. Untreated and HRG treated, respectively). FIG. 4B shows a schematic of the ΔNbox Utrophin/Luciferase construct, the cross depicting the site of deletion mutation removing the N-box. Heregulin does not activate the mutant utrophin promoter bearing a deletion of the N-Box (Error bars=s.e.m., n=36). The differences of expression are not statistically significant.

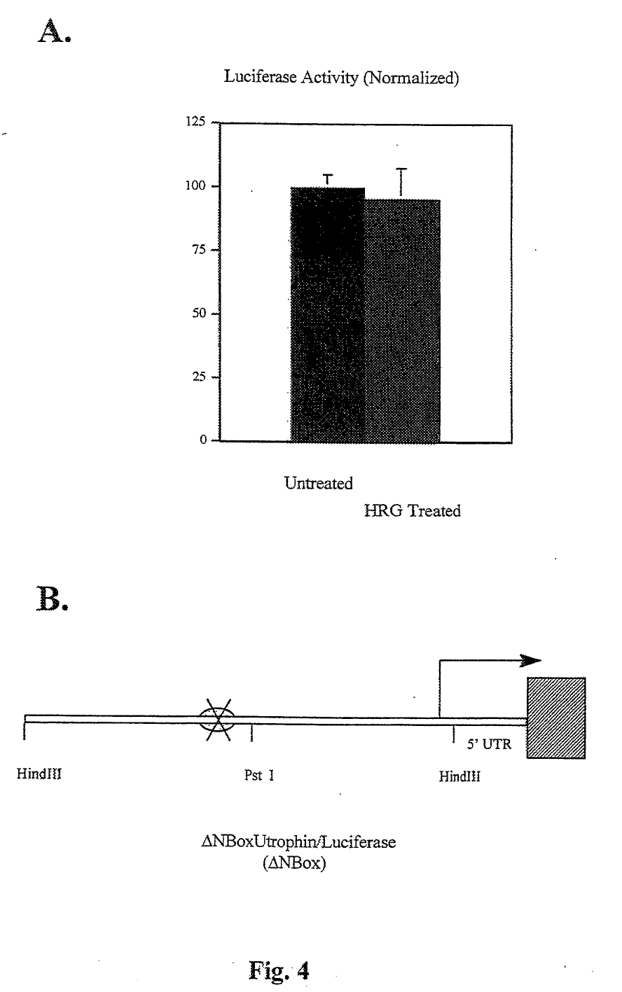

FIG. 5 shows the identification of transcription factors that bind the N-Box of the utrophin promoter. UtroNBox coupled magnetic particles were used to perform DNA-affinity chromatography to purify promoter binding proteins from nuclear extracts of cultured L6 myotubes. 50 μg of nuclear extracts were used and binding proteins eluted in 25 μL of 2M KCL. A 15 μL aliquot was resolved using 12% SDS-PAGE gels and subjected to silver staining (Lane 1) or 5 μL aliquots immunoblotted with affinity purified anti GABPα and GABPβ antibodies and subjected to enhanced chemiluminescence detection (ECL; Lane 2 & 3). Lane 1 (2M KCI fraction) shows silver stained proteins demonstrating that the molecular weight of purified proteins (43 kDa and 58 kDa) exactly matches the molecular weights of GABPβ and GABPα heterodimeric complex of transcription factors. Lane 2 (Anti-GABPα) demonstrates that the 58 kDa band in the purified fraction is recognized by affinity purified anti GABPα antibodies. Lane 3 (Anti-GABPβ) demonstrates that the 43 kDa band in the purified fraction is recognized by affinity purified anti GABPβ antibodies. The additional high molecular weight species presumably represents a GABPβ isoform sharing sequence similarity with GABPβ1 isoform, against which the antibodies were raised. The anti-GABPβ1 antibodies used in this study, are predicted to recognize all GABPβ isoforms.

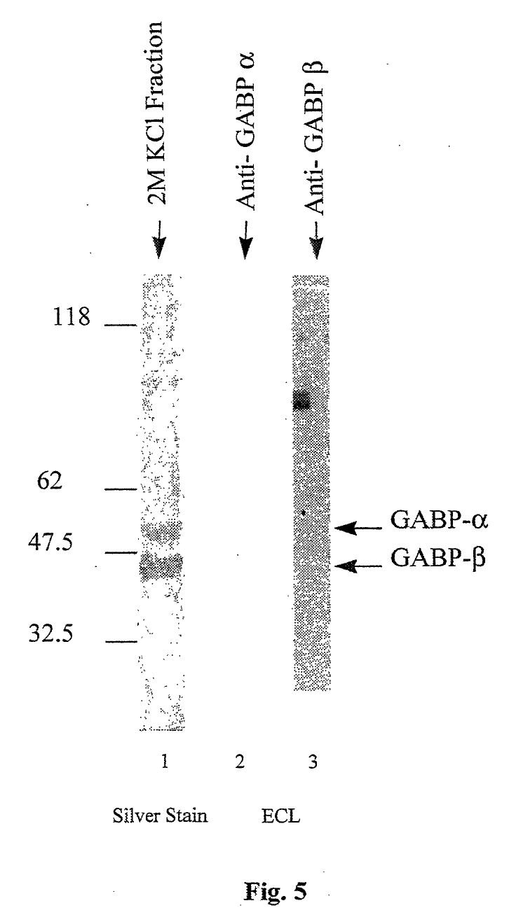

FIG. 6 shows that the utrophin N-Box binds the heterodimeric GABPα/β transcription factor. Electrophoretic mobility shift assay was performed with the radiolabelled oligonucleotide UtroNBox probe and purified GABPα and GABPβ fusion proteins. Lane 1 (Probe+GABPβ) demonstrates the lack of mobility shift using the GABPβ protein, suggesting that the UtroNBox probe does not bind GABPβ by itself. Lane 2 (Probe+GABPα) demonstrates a mobility shift when the probe is incubated with GABPα fusion protein. Lane 3 (Probe+GABPα+Cold probe) shows that the specificity of the interaction with GABPα, since it is competed with a 1000x excess of unlabelled probe. Lane 4 (Probe+GABPα+GABPβ) demonstrates mobility shifts with the formation of GABPα/β multimers when the probe is incubated with both GABPα and GABPβ fusion proteins, suggesting enhancement of GABPα binding by reconstitution of the heterodimeric GABPα/β transcription factor complex.

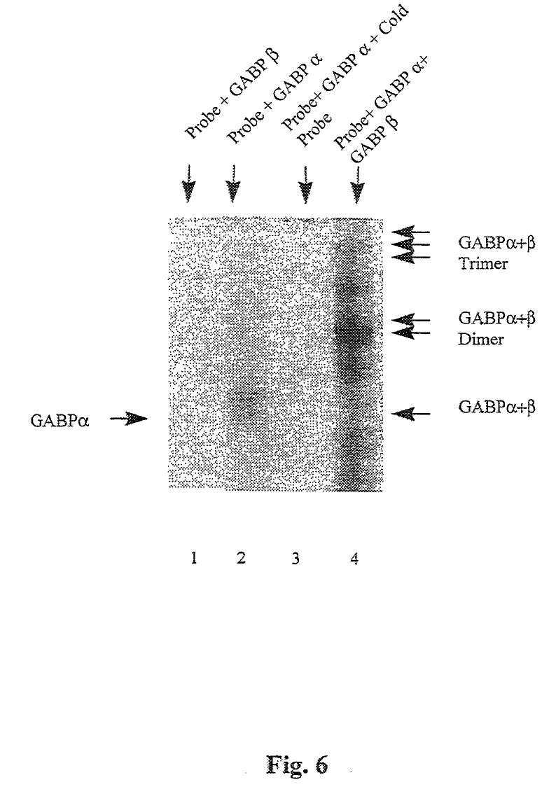

FIG. 7 shows that GABPα/β activates the utrophin promoter in muscle cell cultures (Luciferase Activity (Normalized) vs. Control and Transfected pGABPα+β, respectively). The utrophin promoter luciferase-reporter construct PUBF was co-transfected into L6 muscle cell lines along with expression constructs pGABPα, pGABPβ or pCAGGS (empty vector) along with transfection control pRL and assayed after 24 hours of incubation. PUBF derived firefly luciferase activity is normalized to pRL derived renilla luciferase activity (internal control) in control transfectants as 100%, and expressed as luciferase activity (normalized). The stippled bars represent utrophin promoter activity in cells transfected with empty vector pCAGGS and cross hatched bars the levels in cultures transfected with pGABPα and pGABPβ. GABPα and GABPβ co-transfection increases de novo utrophin transcription in muscle cell cultures to 238% of control levels (Error bars=s.e.m., n=24). Asterisks denote the results were statistically highly significant at p<0.001.

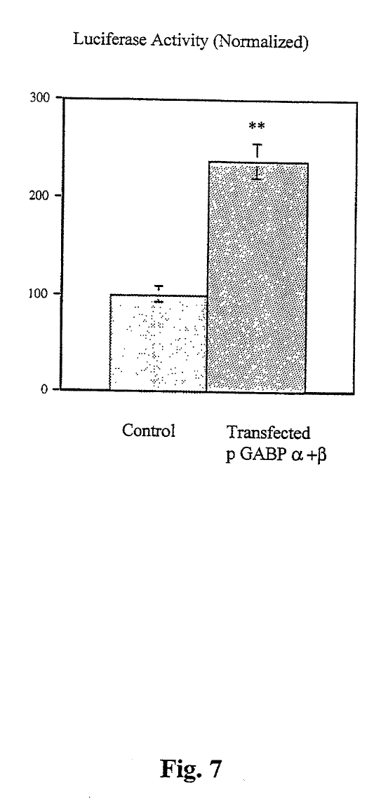

FIG. 8 shows a model for utrophin upregulation by GABPα/β in muscle cell cultures. In this schematic we propose that in unstimulated muscle cultures (and adult muscle) utrophin is transcribed at low levels, possibly because of transcriptional repression activity at the ets-binding site by repressors such as ERF or ERF-like molecules. Upon stimulation with heregulin, transcription is activated via the MAP and PI3 kinase pathways by decreasing the repressor activity, as well as increasing the propensity of GABPα/β transcription factors to bind and heterodimerize, leading to an overall increase of utrophin transcription. The sequence in the figure is from human utrophin promoter and shows the relative position, content and overlap of the N-box (turquoise box) and the site bound by the ets-transcription factor complex GABPα/β (lilac box) in muscle cell cultures, to activate the transcription of utrophin.

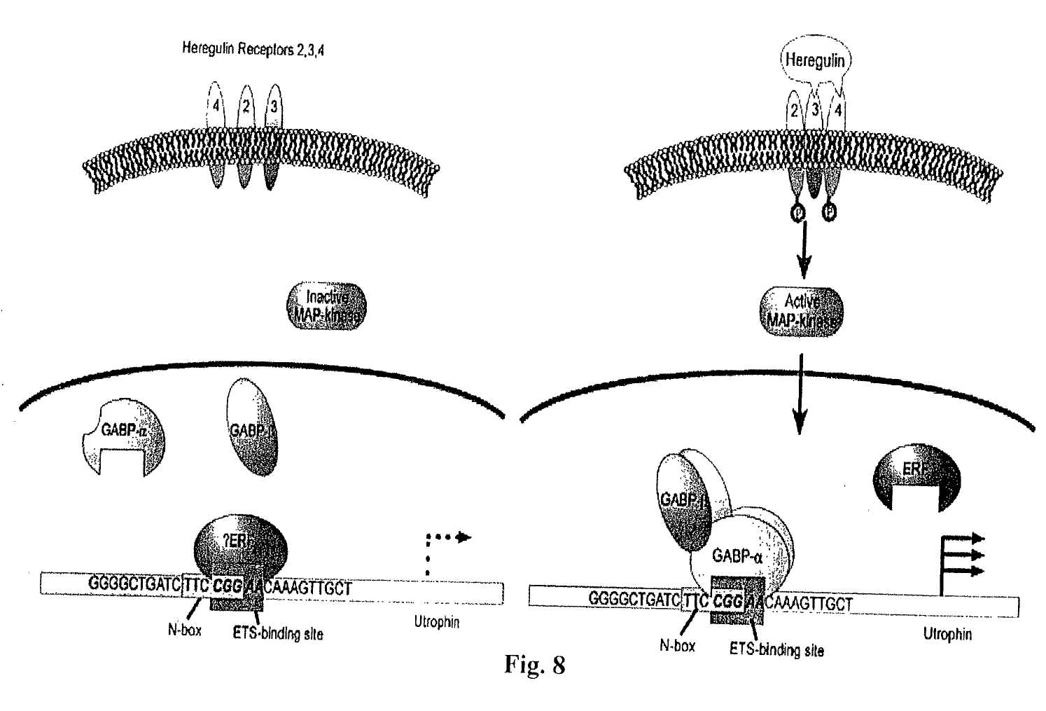

### EXAMPLES

The invention is further illustrated with reference to the following examples which are not intended to be in any way limiting to the scope of the invention as claimed.

### Example 1

In this study we have used DNA-affinity columns, immunoblots, electrophoretic mobility shifts (EMSA) and in vitro expression studies in cultured muscle cells to identify and characterize the mechanism of utrophin regulation at a transcriptional level.

Constructs

The pGABP-α and pGABP-β expression constructs were generated by cloning the human GABP-α and GABP-β1 cDNA's into the mammalian expression vector pCAGGS as described by Rosmarin et al. [Rosmarin A G, Caprio D G, Kirsch D G, Handa H & Simkevich C P: GABP and PU.1 compete for binding, yet cooperate to increase CD18(2 Integrin) transcription; J. Biol. Chem. 1995 270 23627-23633].

The luciferase reporter plasmid pPUBF obtained as described by Dennis et al. [Dennis C L, Tinsley J M, Deconinck A E & Davies K E: Molecular and functional analysis of the utrophin promoter; Nucl. Acids Res. 1996 24 1646-1652] contains the entire human utrophin promoter sequence cloned into the pGL2 basic reporter plasmid.

Additionally, we generated the DNBox construct which has a deletion mutation removing the entire N-box (and core ets-binding site) from the human utrophin promoter and is cloned in the forward orientation in pGL2 Basic vector (Promega, Madison Wis.). The DNBox reporter was generated by cloning the HindIII-SmaI (1-569 bp) fragment of the human utrophin promoter into pGEM3Zf (Promega), generating the plasmid pGEM-L. Next the PstI-HindIII (659-1242 bp) fragment of the promoter was subcloned into pBluescript SKII+yielding pBS-R. The SmaI-PstI fragment (570-658 bp) of the utrophin promoter was used for PCR mutagenesis using the following primers:

| | | |
|---|---|---|
| Nboxless primer | F5′CCCCCCGGGAACGTAGTGGGGCTGATCAACAAAGTTGCTGGGCCGGCGG3′; | (SEQ ID NO: 1); |
| and boxless primer | R5′CCTCCGGCCCGCGCCTCTGCAGCGCTCCGG3′ | (SEQ ID NO: 2). |

The PCR fragment was cloned into a PCR 2.1 vector (Invitrogen, Carlsbad, Calif.) yielding pI. The SmaI-KpnI fragment of pI was subcloned into the pGEM-L yielding pGEM-L+I. Next, the PstI-KpnI fragment of pBS-R was cloned into pGEM-L+I to yield pGEM-L+I+R. The HindIII fragment of pGEM-L+I+R was subcloned into pGL2 Basic to yield the DNBox construct. The clone was sequenced to verify orientation and sequence.

Tissue Culture, Transfections and Luciferase Assays

Primary mouse skeletal muscle cells were generated and propagated essentially as described by Rando & Blau [Rando T A & Blau H M: Primary mouse myoblast purification, characterization, and transplantation for cell-mediated gene therapy; J. Cell. Biol. 1994 125 1275-1287]. The L6 rat muscle cell line was obtained and cultured according to standard protocols (ATCC, Rockville Pike, Md.). For transfections, differentiated muscle cells were plated at 1.2×10⁶ cells per 35 mm well. Plasmids were transfected using Superfect (Qiagen, Hilden, Germany) in case of L6 cells and Calcium phosphate in the case of primary muscle cells.

Cells were transfected with a total of 5 μg DNA/well in case of heregulin response studies, and 7.5 μg DNA/well in case of GABP response studies.

Heregulin (R&D Systems, Minneapolis, Minn.) in PBS was added to achieve a final concentration 1 nM in cultured cells after transfection. In case of the pGABP-α and pGABP-β experiments, 2.5 μg of each plasmid was used and an equivalent amount of either pCAGGS or ssDNA used as a control.

To measure efficiency, we co-transfected pRL control plasmid at a ratio of 1:250 or 1:400. The pRL plasmid (Promega) is designed to express renilla luciferase driven by a cytomegalovirus promoter and was used as an internal control for efficiency of transfection. Luciferase assays were performed using the dual luciferase reagents on a Turner Designs 20/20 luminometer, 24-48 hours after transfection according to instructions supplied by the manufacturer (Promega). Promoter activity values were expressed as normalised luciferase activity, by dividing the firefly luciferase readings with the renilla luciferase reading for each well.

Antibodies and Immunoblots

Human GABP-α and GABP-β1 cDNA's were cloned into the pGEX vector for generation of GABP-α and GABP-β GST fusion proteins [Nuchprayoon I, Simkevich C P, Luo M, Friedman A D & Rosmarin A G: GABP cooperates with c-Myb and C/EBP to activate the neutrophil elastase promoter; Blood 1997 89 4546-4554]. Purified fusion proteins were used as antigens to raise polyvalent antisera in rabbits. Antisera were affinity purified against the appropriate GST fusion protein and negatively selected against GST protein (HTI Bio-Products, Ramona, Calif.), to yield affinity purified GABP-α and GABP-β antibodies. These reagents were used at 1:20000 and 1:2000, respectively. Proteins were detected using enhanced chemiluminescence using the Ultra ECL kit (Pierce, Rockford, Ill.)

Electrophoretic Mobility Shift Assays (EMSA)

Nuclear extracts were prepared from dishes of L6 myotubes cultured to confluence and total proteins quantified, as previously described [Ausubel F et al; Current Protocols in Molecular Biology; Wiley, N.Y., 1995]. The double stranded UtroNBox probe used was 5′ATCTTCcggaa C 3′ (SEQ ID NO: 7) (N-box underlined, ets-domain italicised and lower case) which was end-labelled with g-P³² ATP using T4 Polynucleotide kinase. Typically, radiolabelled probe (1 ng c. 10-100,000 cpm) was incubated on ice for 20 minutes with 10 μg nuclear extracts or 1 μl GST fusion proteins in a 12.5 μL reaction buffer containing 20 mM HEPES pH 7.6, 1 mM MgCl₂, 0.1 mM EGTA, 40 mM KCl, 10% Glycerol, 1 μg ssDNA. Competition with cold probe was performed with pre-incubation with 1000× excess of unlabelled UtroNBox probe. Complexes were resolved by electrophoresis at 200 V for 2 Hours on 4% Acrylamide gels in 25 mM Tris, 192 mM Glycine, 1 mM EDTA (for nuclear extracts) and 12.5 mM Tris, 96 mM Glycine, 0.5 mM EDTA (for fusion proteins) prior to autoradiographic analysis using a Storm Phosphorimager (Molecular Dynamics, Sunnyvale, Calif.).

Quantitative Reverse Transcription-Polymerase Chain Reaction (RT-PCR)

Confluent, differentiated L6 cultures were treated with heregulin (R&D systems) in PBS (final concentration 1 nM) for 30 minutes. Controls were treated with an equivalent volume of PBS (typically 1 μL in 10 mL of culture medium). Bioactivity of heregulin was verified by monitoring increased tyrosine phosphorylation of p185 heregulin receptor species in treated cultures [Fischbach & Rosen, op cit.].

RT-PCR was performed essentially as described by Ausubel, op cit. and Khurana et al. [Khurana, T. S., Watkins, S. C., Chafey, P., Chelly, J., Tome, F. M., Fardeau, M., Kaplan, J. C. & Kunkel, L. M: Immunolocalization and developmental expression of dystrophin related protein in skeletal muscle; Neuromuscul. Disord. 1991 1 185-194]. Briefly, RNA was extracted using Trizol (Life Technologies, Paisly, Scotland) as suggested by the manufacturer. Purified RNA was treated with DNAse and re-purified to exclude DNA contamination. 1 μg RNA was used as template for oligo dT primed reverse transcription (RT) using superscript reverse transcriptase enzyme (Life Technologies). 5% vol/vol of the purified cDNA (corresponding to 50 ng RNA) was used as template for quantitative RT-PCR.

The primers used to amplify a 322 bp fragment of rat utrophin (Accession No. AJ002967; Position 9659-9981) were:

| | |
|---|---|
| RUTROF-5′CAGTATGTGGCCAGAGCACTATGA3′ | (SEQ ID NO: 3); and |
| RUTROR-5′GCAGATTTCTTTGCTCTTCCTCC3′ | (SEQ ID NO: 4). |

As an internal control for efficiency of RT and quantification, we simultaneously amplified a 194 bp fragment of rat glyceraldehyde 3-phosphate dehydrogenase (GAPDH; Accession No. M17701; Position 335-529), using the following primers:

| | |
|---|---|
| RGAPDHF5′CCATGGAGAAGGCTGGGG3′ | (SEQ ID NO: 5); and |
| RGAPDHR5′CAAAGTTGTCATGGATGACC3′ | (SEQ ID NO: 6). |

PCR was performed using a 2 minute denaturation at 94 C. followed by 20 or 25 cycles (for GAPDH and utrophin, respectively) of 94 C. for 30 seconds, 60 C. for 30 seconds, 72 C. for 30 seconds, followed by 72 C. for 7 minutes, conditions that had been optimised for exponential phase amplification of both transcripts. Reactions were also performed in parallel, adding 1 μL P³²dCTP/100 μL reaction mixture, for measuring radioactive incorporation. Products were resolved on 2% Agarose gels and photographed using Ethidium bromide. Photographs were digitised using an Agfa Arcus II scanner at 1600 dpi and bands quantified using ImageQuant 1.1 software for the Mac OS 7.5.3. Radioactive PCR products were resolved on 5% acrylamide gels, dried and the radioactivity incorporated in bands quantified using a Storm Phosphorimager and ImageQuant 1.1 software 10 (Molecular Dynamics). Similar results were obtained in both cases.

DNA-Affinity Purification

The utrophin promoter UtroNBox probe described above, was ligated using T4 DNA ligase to streptavidin magnetic particles that have previously been coupled to a 16 mer oligonucleotide and used for DNA-affinity purification as suggested by the manufacturer (Boehringer Mannheim GmbH, Mannheim, Germany). Typically, 50 μg of L6 myotube nuclear extract was incubated with UtroNBox coupled magnetic particles and eluted in 25 μL of high salt buffer (20 mM HEPES pH 7.6, 1 mM EDTA, 10 mM (NH₄)₂ SO₄, 1 mM DTT, 0.2% Tween (w/v), 2M KCl). The DNA-binding proteins were dialysed to reduce the salt concentration using a 3500 MWCO (Pierce) membrane prior to analysis.

Statistical Analysis

All data were subjected to Student t test for calculation of statistical significance. Where appropriate they were also subjected to an additional parametric test (ANOVA) as well as non parametric (Wilcoxon's rank sum) tests for statistical significance. Statistical analysis was performed using Statview 5.0 (SAS Institute, Cary, N.C.). All data is graphically represented with controls normalised to 100 and increases (or decreases) shown as a % of control levels. Error bars in all cases are s.e.m.

Results

Heregulin Activates Utrophin Expression

In order to address whether heregulin regulates utrophin gene expression, we treated rat L6 myotubes with heregulin and processed the cultures for quantitative RT-PCR.

Differentiated L6 rat myotubes were incubated with 1 nM heregulin in PBS for 30 minutes along with controls. RNA was extracted and quantitative RT-PCR performed (a 322 bp utrophin fragment and a 194 bp GAPDH control fragment was obtained). Heregulin treatment increased the mRNA level of utrophin to 195% compared to control cultures. While this technique is both sensitive and specific, it cannot distinguish whether the observed increase of utrophin mRNA is due to increased utrophin gene transcription or changes in mRNA stability.

To verify the increase in utrophin expression as well as determine whether the increases were indeed due to increased transcription of the utrophin promoter, we transfected murine muscle cultures with a reporter construct containing the entire human utrophin promoter driving the expression of firefly luciferase construct pPUBF [Dennis et al., op cit.].

The utrophin promoter luciferase-reporter construct pPUBF was co-transfected into L6 muscle cell lines or mouse primary muscle cultures along with transfection control plasmid pRL and assayed after 24-48 hours of incubation with heregulin containing medium or control containing medium, heregulin increased the luciferase expression to 138% compared to control cultures. The increased luciferase activity reflects increased de novo transcription of the utrophin promoter in response to heregulin.

Role of N-box in Transcriptional Activation of Utrophin Expression

Having determined that heregulin increased utrophin transcription, we addressed whether the region of the utrophin promoter containing the N-box motif was capable of interacting with putative transcription factors. We performed electrophoretic mobility shift assays (EMSA) to address this issue, using nuclear extracts made from cultured L6 myotubes.

Electrophoretic mobility shift assay was performed with the radiolabelled UtroNBox probe using 10 μg nuclear extract made from cultured L6 myotubes. The UtroNbox probe (from the region of the utrophin promoter containing the N-box motif) binds a factor(s) present in the nuclear extracts of L6 myotubes. The binding is specific since it could be competed with a 1000x excess of unlabelled probe.

The previous experiments suggested that the N-box specifically bound nuclear factor(s) and may be involved in transcriptional regulation. To test whether this region of the promoter mediated heregulin-induced activation, we generated a mutant reporter construct of the human utrophin promoter containing a 6 bp deletion of the N-box motif, called the DN-box construct. L6 muscle cultures were transfected with the DN-box construct and treated with heregulin.

The mutant utrophin promoter luciferase-reporter construct DNBox (deleted in the N-box) was co-transfected into L6 muscle cell lines along with transfection control plasmid pRL and assayed after 24 hours of incubation with either heregulin containing medium or controls. Heregulin did not increase the expression of luciferase in L6 muscle cultures that had been transfected with the DN-box reporter construct, compared to controls. These findings suggest that the N-box region is critical for the heregulin induced transcriptional activation of the utrophin promoter.

GABP-α/β Transcription Factors are Mediators of Utrophin Activation

To identify the transcriptional factors that bind and activate the utrophin promoter in response to heregulin, we made a DNA-affinity column using the UtroNBox probe coupled to magnetic particles. This column was used to purify promoter binding proteins from nuclear extracts of cultured L6 myotubes.

UtroNBox coupled magnetic particles were used to perform DNA-affinity chromatography to purify promoter binding proteins from nuclear extracts of cultured L6 myotubes. 50 μg of nuclear extracts were used and binding proteins eluted in 25 μL of 2M KCL. A 15 μL aliquot was resolved using 12% SDS-PAGE gels and subjected to silver staining or 5 μL aliquots immunoblotted with affinity purified anti GABP-α and GABP-β antibodies and subjected to enhanced chemiluminescence detection. The additional high molecular weight species presumably represents a GABP-β isoform sharing sequence similarity with GABP-β1 isoform, against which the antibodies were raised. The anti-GABP-β1 antibodies used in this study are predicted to recognise all GABP-β isoforms.

Two proteins of Mr 43 kDa and 58 kDa, respectively, were purified from these nuclear extracts. Since, the molecular weights exactly matched members of the ets-related GA-Binding Protein (GABP) a/b transcription factors that have been implicated in case of the nACHR genes [Sapru M K, Florance S K, Kirk C & Goldman D: Identification of a neuregulin and protein-tyrosine phosphatase response element in the nicotinic acetylcholine receptor e subunit gene: Regulatory role of an ets transcription factor; Proc. Natl. Acad. Sci. U.S.A. 1998 95 1289-1294; Schaeffer L, Duclert N, Dymanus M H & Changeux J P: Implication of a multisubunit Ets-related transcription factor in synaptic expression of the nicotinic acetylcholine receptor; EMBO J. 1998 17 3078-3090], we performed immunoblot analysis using affinity-purified antibodies against GABP-α and GABP-β on the purified fractions [Brown T A & McKnight S L: Specificities of protein-protein and protein-DNA interaction of GABP alpha and two newly defined ets-related proteins; Genes Dev. 1992 6 2502-2512; de la Brousse F, Birkenmeier E H, King D S, Rowe L B & McKnight S L: Molecular and genetic characterization of GABP beta; Genes Dev. 1994 8 1853-1865], to determine the molecular identity of the purified protein. The 43 kDa and 58 kDa proteins were indeed recognised by antibodies specific for GABP-β and GABP-α transcription factors.

To independently verify the role of GABP-α/β transcription factors in heregulin mediated transcriptional activation, we asked whether the region of utrophin promoter containing the N-box motif was capable of specifically interacting with these transcription factors by performing EMSA with GABP-α/β fusion proteins [Rosmarin A G, Caprio D G, Kirsch D G, Handa H & Simkevich C P: GABP and PU.1 compete for binding, yet cooperate to increase CD18 (2 Integrin) transcription; J. Biol. Chem. 1995 270 23627-23633].

Electrophoretic mobility shift assay was performed with the radiolabelled oligonucleotide UtroNBox probe and purified GABP-α and GABP-β fusion proteins.

These experiments showed that the UtroNBox probe bound specifically to GABP-α, as it could be competed with an excess of unlabelled probe. As predicted by structural studies of GABP complex [Brown & McKnight, op cit.; de la Brousse et al., op cit.], the UtroNBox probe did not bind GABP-β alone. However, bound the in vitro reconstituted heterodimeric complex of GABP-α/β with enhanced efficiency as evidenced by the formation of multimeric complexes of the UtroNBox probe with GABP-α/β.

Having determined that the GABP-α/β complex was biochemically associated with the N-box region of the utrophin promoter, we tested whether the GABP-α/β can functionally activate the promoter and increase utrophin transcription. We transfected L6 myotubes with pPUBF, along with expression constructs encoding the GABP-α and GABP-β [Dennis et al., op cit.; Rosmarin et al., 1995, op cit.]. Control cultures were transfected with the reporter construct along with either empty vector pCAGGS, or an equivalent amount of ssDNA.

The utrophin promoter luciferase-reporter construct PUBF was co-transfected into L6 muscle cell lines along with expression constructs pGABP-α, pGABP-β or pCAGGS (empty vector) along with transfection control pRL and assayed after 24 hours of incubation. PUBF derived firefly luciferase activity is normalised to pRL derived renilla luciferase activity (internal control) in control transfectants as 100%, and expressed as luciferase activity (normalised).

GABP-α and GABP-β co-transfection increases de novo utrophin transcription in muscle cell cultures to 238% of control levels. The increased luciferase activity reflects transcriptional activation of the utrophin promoter by the heterodimeric complex of GABP-α and GABP-β, in muscle cells. These data demonstrate that transfection with GABP-α/β increases the de novo transcription of the utrophin gene.

Utrophin Upregulation by GABP-α/β in Muscle Cell Cultures

Based on the above experiments we assume that in unstimulated muscle cultures (and adult muscle) utrophin is transcribed at low levels, possibly because of transcriptional repression activity at the ets-binding site by repressors such as ERF or ERF-like molecules. Upon stimulation with heregulin, transcription is activated via the MAP and PI3 kinase pathways [Tansey M G, Chu G C & Merlie J P: ARIA/HRG regulates the AChR e subunit gene expression at the neuromuscular synapse via activation of the phosphatidylinositol 3-kinase and RAS/MAPK pathway; J. Cell. Biol. 1996 134 465-476] by decreasing the repressor activity, as well as increasing the propensity of GABP α/β transcription factors to bind and heterodimerize, leading to an overall increase of utrophin transcription.

1. A pharmaceutical composition, comprising:
a therapeutically effective amount of a GA Binding Protein (GABP); and
one or more pharmaceutically acceptable adjuvant, excipient, carrier, buffer, diluent and/or other customary pharmaceutical auxiliary.

2. The pharmaceutical composition according to claim 1, in which the GA Binding protein (GABP) is GABPα and/or GABPβ.

3. A method for treatment or alleviation of diseases, disorders or conditions relating to muscular dystrophia in a living body, said method comprising:
administering to said living body an effective amount of a GA binding Protein (GABP).

4. The method according to claim 3, wherein the GA Binding Protein (GABP) is GABPα and/or GABPβ.

5. The method according to claim 3 or 4, in which the disease, disorder or condition relating to muscular dystrophia is Duchenne's muscular dystrophy (DMD).

6. A method for upregulation of utrophin gene expression at the neuromuscular junction of skeletal muscle in a living body, said method comprising:
administering to said living body an effective amount of a GA Binding Protein (GABP).

7. The method according to claim 6, wherein GA Bining Protein (GABP) is GABPα and/or GABPβ.

8. The method of claim 6, for treatment or alleviation of diseases, disorders or conditions relating to muscular dystrophia.

9. The method of claim 8, wherein the muscular dystrophia is Duchenne's muscular dystrophy (DMD).

10. A method for the upregulation of utrophin gene expression at the neuromuscular junction of skeletal muscle in a living body, said method comprising:
administering to said living body an effective amount of a neurite derived growth factor.

11. The method of claim 10, wherein the neurite derived growth factor is heregulin, NDF, ARIA, or GGF.

12. The method of claim 10, wherein the neurite derived growth factor is an activator of HER/ErbB class of receptors, preferably ErbB-2, ErbB-3, or ErbB-4.

13. The method of claim 10, wherein the neurite derived growth factor is heregulin, a heregulin-like polypeptide, an isoform of heregulin, or a variant of heregulin.

14. The method of claim 13, wherein the heregulin polypeptide is heregulin-α (HRG-α), heregulin-β1 (HRG-β1), heregulin-β2 (HRG-β2) or heregulin-β3 (HER-β3).

15. The method of claim 10, for treatment or alleviation of diseases, disorders or conditions relating to muscular dystrophia.

16. The method of claim 15, wherein the muscular dystrophia is Duchenne's muscular dystrophy (DMD).


SEQUENCE LISTING (7 sequences)

SEQ ID NO: 1 - DNA, 49 bp, Artificial Sequence
  PCR Primer derived from the human utrophin promoter
ccccccgggaacgtagtggggctgatcaacaaagttgctgggccggcgg

SEQ ID NO: 2 - DNA, 30 bp, Artificial Sequence
  PCR Primer derived from the human utrophin promoter
cctccggcccgcgcctctgcagcgctccgg

SEQ ID NO: 3 - DNA, 24 bp, Artificial Sequence
  PCR Primer derived from rat utrophin
cagtatgtggccagagcactatga

SEQ ID NO: 4 - DNA, 23 bp, Artificial Sequence
  PCR Primer derived from rat utrophin
gcagatttctttgctcttcctcc

SEQ ID NO: 5 - DNA, 18 bp, Artificial Sequence
  PCR Primer derived from rat glyceraldehyde 3-phosphate dehydrogenas
ccatggagaaggctgggg

SEQ ID NO: 6 - DNA, 20 bp, Artificial Sequence
  PCR Primer derived from rat glyceraldehyde 3-phosphate dehydrogenas
caaagttgtcatggatgacc

SEQ ID NO: 7 - DNA, 12 bp, Artificial Sequence
  double stranded UtroNBox probe
atcttccggaac